(19) United States
(12) Patent Application Publication
(10) Pub. No.: US 2008/0242872 A1
(43) Pub. Date: Oct. 2, 2008

(54) AMORPHOUS CARVEDILOL PHOSPHATE AND METHOD OF MANUFACTURING THEREOF

(75) Inventors: Jerzy DRYGAS, Krotoszyn (PL); Jolanta PAWLOWSKA, Starogard Gdanski (PL); Roman SZRAMKA, Gniew (PL); Jan RAMZA, Pruszkow (PL)

Correspondence Address: MATTHIAS SCHOLL, 14781 MEMORIAL DRIVE, SUITE 1319, HOUSTON, TX, 77079, US

(73) Assignee: ZAKLADY FARMACEUTYCZNE POLPHARMA S.A., Starogard Gdanski (PL)

(21) Appl. No.: 11/866,418

(22) Filed: Oct. 3, 2007

(30) Foreign Application Priority Data
Mar. 28, 2007 (PL) PL382084

(51) Int. Cl. C07D 209/82

(52) U.S. Cl. 548/444

(57) ABSTRACT

The subject of the invention is amorphous carvedilol phosphate and a method of manufacturing amorphous carvedilol phosphate comprising removing solvent from carvedilol phosphate solution by means of spray drying.

### CROSS-REFERENCE TO RELATED APPLICATIONS

This application claims priority to Polish Patent Application No. PL382084 filed on Mar. 28, 2007, the contents of which are incorporated herein by reference.

### BACKGROUND OF THE INVENTION

1. Field of the Invention

This invention relates to amorphous carvedilol phosphate, and a method of manufacturing amorphous form of carvedilol phosphate.

2. Description of the Related Art

(±)1-(9H-carbazol-4-yloxy)-3-[2-(2-methoxyphenoxy)ethylamino]propan-2-ol,

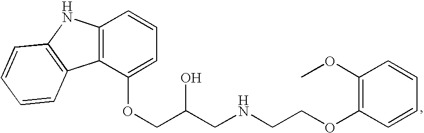

known under its generic name as carvedilol, is a compound valued for its unique action mechanism combining non-selective inhibition of the β-adrenergic receptors in the heart, resulting in a decrease in blood pressure, heart rate and stroke volume, with the inhibition of the α1-adrenergic receptors, resulting in dilation of the blood vessels and decrease in the systemic vascular resistance.

The synthesis of an entire family of crystalline carbazolyl-4-oxy-propanolamine derivatives as well as their pharmaceutically acceptable salts, including carvedilol, was first disclosed in the European Pat. App. No. 0 004 920.

Although carvedilol and its derivatives are used in the treatment of hypertension and angina pectoris, their application is somewhat limited by their poor water solubility.

Poorly water-soluble substances show a better solubility when in dispergated form, resulting in a better bioavailability in comparison to their crystalline equivalents.

Amorphous forms can be obtained using several methods such, as solvent vaporization, solvent precipitation, lyophilization, spray-drying and spray congelation.

In general, however, amorphous forms are physically and chemically less stable from the crystalline forms, molecules of which are aligned thus demonstrating a lower inner energy.

In light of the foregoing, a novel form of carvedilol with greater aqueous solubility, chemical stability, etc. would offer many potential benefits for provision of medicinal products containing the drug carvedilol. Such benefits would include products with the ability to achieve desired or prolonged drug levels in a systemic system by sustaining absorption along the gastrointestinal tract of mammals (i.e., such as humans), particularly in regions of neutral pH, where a drug, such as carvedilol, has minimal solubility.

### SUMMARY OF THE INVENTION

It was unexpectedly discovered that stable carvedilol phosphate in amorphous form can be obtained in a way that is economical and devoid of the shortcomings of previous literature methods.

In one embodiment, the invention provides amorphous carvedilol phosphate.

In certain classes of this embodiment, the X-ray powder diffraction spectrum of amorphous carvedilol phosphate lacks discemable acute peaks.

In certain classes of this embodiment, the X-ray powder diffraction spectrum of amorphous carvedilol phosphate obtained with a Cu K-alpha radiation contains only very broad characteristic peaks at about 6 (2θ) and 22.5 (2θ) in the range between 0 and 40 (2θ).

In certain classes of this embodiment, the infrared spectrum of amorphous carvedilol phosphate has sharp bands at about 3405, 3060, 2362, 1627, 1606, 1587, 1505, 1455, 1401, 1348, 1334, 1306, 1255, 1217, 1178, 1125, 1101, 1021, 945, 786, 474, 723, 512 cm⁻¹.

In certain classes of this embodiment, the X-ray powder diffraction spectrum of amorphous carvedilol phosphate obtained with a Cu K-alpha radiation is substantially as illustrated in FIG. 2

In certain classes of this embodiment, the infrared spectrum of amorphous carvedilol phosphate is substantially as illustrated in FIG. 1.

In other aspects the invention provides a method of manufacturing amorphous carvedilol phosphate comprising removing solvent from carvedilol phosphate solution.

In certain classes of this embodiment, the solvent is removed by means of a spray dryer.

In certain classes of this embodiment, the solvent is removed by spray drying.

In certain classes of this embodiment, the solvent is methanol.

In another embodiment, the invention provides a method of manufacturing amorphous carvedilol phosphate comprising: (a) producing a mist of a carvedilol phosphate solution comprising carvedilol phosphate, a solvent, and a gas; and (b) vaporizing and removing the solvent by means of a drying gas.

In a class of this embodiment, the solvent is selected from methanol, ethanol, n-propanol, 2-propanol, their mixtures or their mixtures with water.

In a class of this embodiment, the gas is nitrogen, argon, or helium, and the drying gas is nitrogen, argon, or helium.

In a class of this embodiment, the mist of carvedilol phosphate solution in (a) has a temperature between 25 and 100° C.

In a class of this embodiment, vaporization of the solvent in (b) occurs at 120 to 175° C.

### BRIEF DESCRIPTION OF THE DRAWINGS

The objects and advantages of the invention will become more readily apparent after reading the ensuing descriptions of the non-limiting illustrative embodiment and viewing the accompanying drawings, in which

FIG. 1 is an FT-IR spectrum of a typical lot of amorphous state carvedilol obtained by methods of the invention; and

FIG. 2 is an XRPD (x-ray powder diffraction) pattern thereof.

### DETAILED DESCRIPTION OF THE INVENTION

Amorphous carvedilol phosphate is obtained through a very fast solvent vaporization of carvedilol phosphate solution, e.g., in a spray dryer apparatus comprising:

a container for the carvedilol phosphate solution,

a pump passing the solution to the atomiser,

a heater of the gas fed into the atomiser,

a heater of the drying gas fed into the drying chamber,

an atomiser producing a mist of the carvedilol phosphate solution by mixing the carvedilol phosphate solution and a gas,

a drying chamber where the process of the solvent vaporization takes place,

pipes feeding the product dust (amorphous carvedilol phosphate) to a separation unit for separating product from the drying gas,

a cyclone separator with a product receptacle where the separation of the gas and the product takes place,

a filter purifying the used drying gas of the product remains, and

a unit for regeneration of the drying gas (outdropping solvents or absorption).

Organic solvents, such as aliphatic monohydric alcohols: methanol, ethanol, n-propanol, 2-propanol, their mixtures and/or their mixtures with water are used to prepare the carvedilol phosphate solution.

Carvedilol phosphate solution is obtained by dissolving crystalline carvedilol phosphate in an organic solvent, mixture of organic solvents or their mixture with water, or by dissolving carvedilol in an organic solvent, mixture of organic solvents, or their mixture of one or more organic solvents with water and adding an equimolar amount of phosphoric acid (e.g., 70-85% aqueous solution of H₃PO₄).

Concentrations of carvedilol phosphate solutions fed into the atomiser at room temperature range from 0.1 to 2.9% w/v. The temperatures of the heated solutions range from 25° C. to 100° C. and their concentrations range from 2.5-20% w/v.

The carvedilol phosphate solution is fed into the atomiser at a velocity ranging from 1 to 1,000 mL/min.

The gas fed into the atomiser and the drying gas is air or an inert gas such, as: nitrogen, argon, or helium.

The temperature of the gas fed into the atomiser ranges from 25 to 100° C.

The temperature of the drying gas fed into the drying chamber ranges from 120 to 175° C.

The drying gas is fed into the drying chamber concurrently with carvedilol phosphate aerosol.

The temperature of the waste gas (used drying gas) after exiting the cyclone ranges from 35 to 120° C.

The usage of the drying gas and the atomiser gas ranges from 10 to 200 Nm³/h.

The process of efficiency ranges from 80 to 98%.

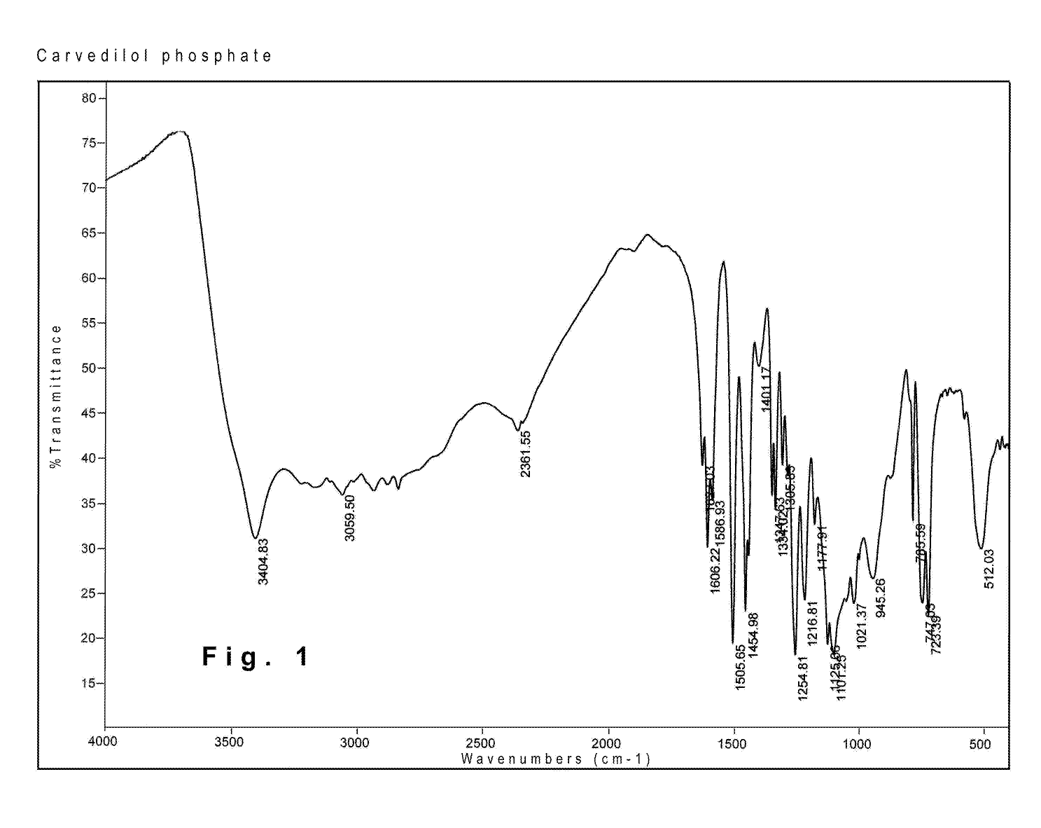

The obtained product is examined using the X-ray diffraction method and infrared spectrometry. An exemplary spectrometric image is shown in FIG. 1. The infrared spectrum shows sharp bands at about 3405, 3060, 2362, 1627, 1606, 1587, 1505, 1455, 1401, 1348, 1334, 1306, 1255, 1217, 1178, 1125, 1101, 1021, 945, 786, 474, 723, and 512 cm⁻¹. An exemplary image from the diffraction examination is shown in FIG. 1. The X-ray powder diffraction spectrum obtained with a Cu K-alpha radiation lacks discemable acute peaks and contains only very broad characteristic peaks at about 6 (2θ) and 22.5 (2θ) in the range between 0 and 40 (2θ). Both spectrochemical analyses confirm that the examined product is amorphous.

### EXAMPLES

The following spray dryers were used in the below-mentioned examples: (1) MOBILE MINOR™ Spray Dryer (GEA Niro Inc.) in closed-cycle; and (2) Anhydro MicraSpray 150 CC (Anhydro A/S).

### Example 1

1.0 kg of crystalline carvedilol phosphate was dissolved in 40 dm³ of methanol at room temperature. The solution was fed into the atomiser through a peristaltic pump with the velocity of between 30 and 50 mL/min. The nitrogen fed into the atomiser was heated to the temperature of 63±2° C. The temperature of the nitrogen fed into the drying chamber was set to within the range of from 155 to 165° C. The product, amorphous carvedilol phosphate, was received at the exit of the cyclone separator in the amount of from 40 to 70 g/h. After 16-18 hours of work the prepared solution of carvedilol phosphate was used up and 950 g of amorphous product was obtained.

### Example 2

1.0 kg of crystalline carvedilol phosphate was dissolved in 10 dm³ of methanol at the temperature of 50° C. The solution was fed into the atomiser through a peristaltic pump with the velocity of between 20 and 30 mL/min. The nitrogen fed into the atomiser was heated to the temperature of 63±2° C. The temperature of the nitrogen fed into the drying chamber was set to within the range of between 155 and 165° C. The product, amorphous carvedilol phosphate, was received at the exit of the cyclone separator in the amount of between 100 and 180 g/h. After 7-9 hours of work the prepared solution of carvedilol phosphate was used up and 920 g of amorphous product was obtained.

What is claimed is:

1. Amorphous carvedilol phosphate.

2. The amorphous carvedilol phosphate of claim 1, characterized in that its X-ray powder diffraction spectrum lacks discemable acute peaks.

3. The amorphous carvedilol phosphate of claim 1, characterized in that its X-ray powder diffraction spectrum obtained with a Cu K-alpha radiation contains only very broad characteristic peaks at about 6 (2θ) and 22.5 (2θ) in the range between 0 and 40 (2θ).

4. The amorphous carvedilol phosphate of claim 1, characterized by an infrared spectrum having sharp bands at about 3405, 3060, 2362, 1627, 1606, 1587, 1505, 1455, 1401, 1348, 1334, 1306, 1255, 1217, 1178, 1125, 1101, 1021, 945, 786, 474, 723, 512 cm⁻¹.

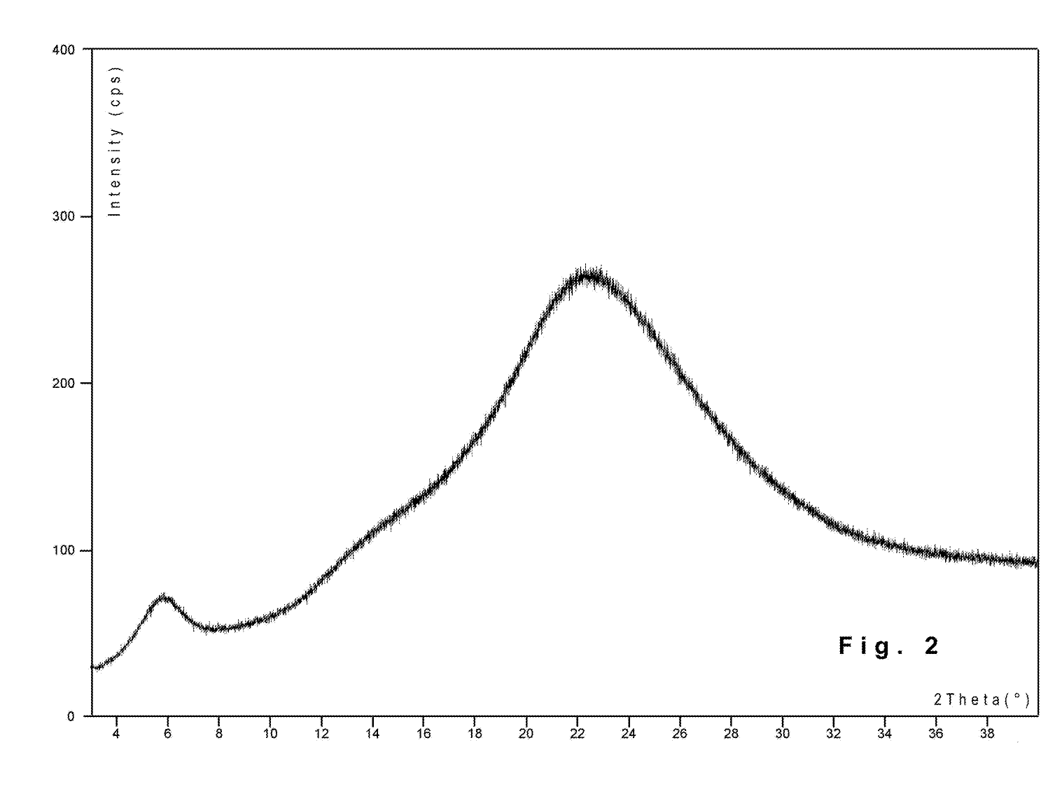

5. The amorphous carvedilol phosphate of claim 1 having an X-ray powder diffraction spectrum obtained with a Cu K-alpha radiation substantially as illustrated in FIG. 2.

6. The amorphous carvedilol phosphate of claim 1 having an infrared transmittance spectrum substantially as illustrated in FIG. 1.

7. A method of manufacturing amorphous carvedilol phosphate comprising removing solvent from carvedilol phosphate solution.

8. The method of claim 7, wherein the solvent is removed by means of a spray dryer.

9. The method of claim 7, wherein the solvent is removed through spray drying.

10. The method of claim 7, wherein the solvent is methanol.

11. A method of manufacturing amorphous carvedilol phosphate comprising
(a) producing a mist of a carvedilol phosphate solution comprising carvedilol phosphate, a solvent, and a gas;
(b) vaporizing and removing the solvent by means of a drying gas.

12. The method of claim 11, wherein the solvent is selected from methanol, ethanol, n-propanol, 2-propanol, their mixtures or their mixtures with water.

13. The method of claim 11, wherein the gas is nitrogen, argon, or helium, and the drying gas is nitrogen, argon, or helium.

14. The method of claim 11, wherein the mist of carvedilol phosphate solution in (a) has a temperature between 25 and 100° C.

15. The method of claim 11, wherein vaporization of solvent in (b) occurs at 120 to 175° C.
